(19) United States
(12) Patent Application Publication
(10) Pub. No.: US 2022/0287631 A1
(43) Pub. Date: Sep. 15, 2022

(54) SYSTEM AND METHOD OF MONITOR SLEEP

(71) Applicant: WeSense Healthcare Ltd., Rishon LeTsiyon (IL)

(72) Inventors: Miki Peer, Yavne (IL); Madoc El Mali, Rishon LeTsiyon (IL)

(73) Assignee: WeSense Healthcare Ltd.

(21) Appl. No.: 17/195,705

(22) Filed: Mar. 9, 2021

(51) Int. Cl. A61B 5/00; A61B 5/0205; A61M 21/00; G16H 40/67; G16H 50/30; G16H 50/70; G06N 5/04; G06N 20/00

(52) U.S. Cl. CPC A61B 5/4818; A61B 5/7275; A61B 5/7267; A61B 5/4812; A61B 5/0205; A61B 5/746; A61M 21/00; A61B 5/4836; G16H 40/67; G16H 50/30; G16H 50/70; G06N 5/04; G06N 20/00; A61B 5/024

(57) ABSTRACT

A system, a wearable device, and a method for monitoring a sleep cycle of an infant. The system and/or the wearable device are configured to vary the monitoring rate based on the sleep stages and awake the infant and/or send an alarm when the infant is in a life-threatening condition.

### TECHNICAL FIELD

Some embodiments described herein are generally related to infant sleep cycle monitoring and, more particularly, to sudden and unexpected infant deaths (SUIDs).

### BACKGROUND

Sudden and Unexpected Infant Deaths (SUIDs) may include 80% of Sudden Infant Death Syndrome (SIDS), and the other 20% cases are often caused by infections, genetic disorders, and heart problems. SIDS is known as cot death, or crib death, is the sudden unexplained death of a child of less than one year of age. Diagnosis requires that the death remain unexplained even after a thorough autopsy and detailed death scene investigation. SIDS usually occurs during sleep.

Most SIDS deaths happen in babies at the age between one month and four months old, and the majority (90%) of SIDS deaths may occur before a baby reaches six months of age. However, SIDS deaths can happen anytime during a baby's first year. Thus, there is a need to prevent and protect the infant from crib death.

### SUMMARY

Embodiments related to a system, a method, and a product for monitoring a sleep cycle of an infant are described hereinbelow by the ways of example only.

One embodiment may include a system for monitoring a sleeping behavior of an infant, comprising a wearable device comprises processing circuitry, wherein the processing circuitry is configured to: determine an infant risk profile for sudden infant death syndrome (SIDS) based on an indication generated by a predictive Artificial Intelligent (AI) algorithm; perform in parallel a first periodically monitoring having a first monitoring schedule of one or more infant vital signs to set a risk threshold value, by monitoring one or more sensors that are operably coupled to the wearable device and a second periodically monitoring having a second monitoring schedule to determine a deep sleep stage based on at least two vital signs; at the deep stage and Rapid Eye Movement (REM) sleep stage, adjust the first monitoring schedule and update the risk threshold value base on the infant risk profile and the one or more vital signs; and when the risk threshold value crossed, perform a third monitoring at a third monitoring schedule to predict a life-threatening state; and at the life-threatening state, generate one or more signals to prevent the infant from being at the life-threatening state.

For example, the processing circuitry is configured to determine the infant risk profile based on a machine learning algorithm, wherein the machine learning algorithm is configured to: weight data received from the two or more sensors to generate weighted data; compare a historical medical measurement data of a plurality of infants to a to the weighted data received from the one or more sensor, and set a risk level of the infant based on the comparison.

In the one embodiment, for example, the machine learning algorithm is configured to identify a sleep stage based on the two or more vital signs; and predict an entering to a next sleep stage base on the two or more vital signs, wherein the two or more vital signs comprise a heart rate pattern and historical sleep pattern of the infant.

In the one embodiment, for example, the machine learning algorithm is configured to: predict a life-threatening state of the infant based on historical databased records, one or more vital signs of the infant.

In the one embodiment, for example, the machine learning algorithm is configured to: analyze data received from the one or more vital signs and from a historical database; and define the risk threshold value based on the learning.

The one embodiment, for example, comprises a server, wherein the server is configured to perform the predictive AI algorithm according to the machine learning algorithm.

In the one embodiment, for example, the first monitoring schedule is set based on an awareness state of the infant.

In the one embodiment, for example, the two or more vital signs comprise at least an infant movement, and a heart rate pattern and the processing circuitry is configured to: identify a sleep stage of the infant based on a combination of the movement of the infant and the heart rate pattern of the infant.

In the one embodiment, for example, the processing circuitry is configured to: determine an infant lying position based on an infant movement indication.

In the one embodiment, for example, the one or more signals comprise a signal configured to vibrate the infant in a low frequency.

In the one embodiment, for example, the one or more signals comprise a signal configured to cause a brain stimulation to the infant.

In the one embodiment, for example, the one or more signals comprise: a signal configured to transmit a wave, wherein the frequency of the wave is determined according to the infant profile.

In the one embodiment, for example, the one or more signals comprise: a signal configured to cause an alarm at an alarm device.

One other embodiment may include a product comprising one or more tangible computer-readable non-transitory storage media comprising program instructions for, wherein execution of the program instructions by one or more processors comprising: determining an infant risk profile for sudden infant death syndrome (SIDS) based on an indication generated by a predictive Artificial Intelligent (AI) algorithm; performing in parallel a first periodically monitoring having a first monitoring schedule of one or more infant vital signs to set a risk threshold value, by monitoring one or more sensors that are operably coupled to the wearable device and a second periodically monitoring having a second monitoring schedule to determine a deep sleep stage based on at least two vital signs; at the deep stage and Rapid Eye Movement (REM) sleep stage, adjusting the first monitoring schedule and update the risk threshold value base on the infant risk profile and the one or more vital signs; when the risk threshold value crossed, performing a third monitoring at a third monitoring schedule to predict a life-threatening state; and at the life-threatening state, generating one or more signals to prevent the infant from being at the life-threatening state.

For example, the execution of the program instructions by one or more processors comprises execution of a machine learning program instructions, wherein execution of the machine learning program instructions by one or more processors at a server comprising: weighting data received from the two or more sensors to generate weighted data; comparing a historical medical measurement data of a plurality of infants to a to the weighted data received from the one or more sensor, and setting a risk level of the infant based on the comparison.

In the one other embodiment, for example, the execution of the machine learning program instructions by one or more processors comprising: identifying a sleep stage based on the two or more vital signs; and predicting an entering to a next sleep stage base on the two or more vital signs, wherein the two or more vital signs comprise a heart rate pattern and historical sleep pattern of the infant.

In the one other embodiment, for example, the execution of the machine learning program instructions by one or more processors comprising: analyzing data received from the one or more vital signs and from a historical database, and defining the risk threshold value based on the learning.

In the one other embodiment, for example, the execution of the program instructions by one or more processors comprising: setting the first monitoring schedule based on an awareness state of the infant.

In the one other embodiment, for example, the two or more vital signs comprise at least an infant movement, and a heart rate pattern and the execution of the program instructions by one or more processors comprise: identifying a sleep stage of the infant based on a combination of the movement of the infant and the heart rate pattern of the infant.

In the one other embodiment, for example, the execution of the program instructions by one or more processors comprising: determining an infant lying position based on an infant movement indication.

It is understood from the present disclosure described a solution for shortcomings in the field of the art. More specifically, the embodiments described herein enable to predict by a predictive AI algorithm a life-threatening state of the infant while sleeping. The prediction is made by monitoring at different monitoring schedules the infant vital signs base on the sleeping stage of the infant, e.g., N1, N2, N3 (deep sleep), and REM, and using machine learning to analyze the vital signs data to identify the life-threatening state.

### BRIEF DESCRIPTION OF THE DRAWING

FIG. 1 illustrates a block diagram of a system to monitor a sleep of an infant, according to some demonstrative embodiments.

FIG. 2 illustrates a flow chart of a method to monitor a sleep of an infant, according to some demonstrative embodiments.

FIG. 3 illustrates a block diagram of a wearable device configured to monitor a sleep of an infant, according to some demonstrative embodiments.

FIG. 4 illustrates a bracelet configured to monitor the vital signs of an infant, according to some demonstrative embodiments.

FIG. 5 illustrates a product of manufacture, according to some demonstrative embodiments.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units, and/or circuits have not been described in detail so as not to obscure the discussion.

Discussions made herein utilizing terms such as, for example, “processing,” “computing,” “calculating,” “determining,” “establishing,” “analyzing,” “checking,” or the like, may refer to operation(s) and/or process(es) of a computer, a computing platform, a computing system, or other electronic computing devices, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

The terms “plurality” and “a plurality,” as used herein, include, for example, “multiple” or “two or more.” For example, “a plurality of items” includes two or more items.

References to “one embodiment,” “an embodiment,” “demonstrative embodiment,” “various embodiments,” etc., indicate that the embodiment(s) so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, unless otherwise specified, the use of the ordinal adjectives “first,” “second,” “third,” etc., to describe a common object merely indicate that different instances of like objects are being referred to and are not intended to imply that the objects so described must be in a given sequence, either temporally, spatially, in ranking, or any other manner.

As used herein, the term “circuitry” may refer to, be part of, or include, an Application Specific Integrated Circuit (ASIC), an integrated circuit, an electronic circuit, a processor (shared, dedicated, or group), and/or memory (shared, dedicated, or group), that execute one or more software or firmware programs, a combinational logic circuit, and/or other suitable hardware components that provide the described functionality. In some demonstrative embodiments, the circuitry may be implemented in, or functions associated with the circuitry may be implemented by one or more software or firmware modules. In some demonstrative embodiments, the circuitry may include logic, at least partially operable in hardware.

The term “logic” may refer, for example, to computing logic embedded in the circuitry of a computing apparatus and/or computing logic stored in a memory of a computing apparatus. For example, the logic may be accessible by a processor of the computing apparatus to execute the computing logic to perform computing functions and/or operations. In one example, logic may be embedded in various types of memory and/or firmware, e.g., silicon blocks of various chips and/or processors. Logic may be included in and/or implemented as part of various circuitry, e.g., radio circuitry, receiver circuitry, control circuitry, transmitter circuitry, transceiver circuitry, processor circuitry, and/or the like. In one example, logic may be embedded in volatile memory and/or non-volatile memory, including random access memory, read-only memory, programmable memory, magnetic memory, flash memory, persistent memory, and the like. Logic may be executed by one or more processors using memory, e.g., registers, stuck, buffers, and/or the like, coupled to the one or more processors, e.g., as necessary to execute the logic.

As used herein, the term “module” as used hereinbelow is an object file that contains code to extend the running kernel environment.

As used herein, the term “database” “database” refers to a set of related data and the way it is organized. Access to this data may be provided by a “database management system” (DBMS) consisting of an integrated set of computer software that allows users to interact with one or more databases and provides access to all of the data contained in the database.

As used herein, the term “Artificial intelligence (AI)” is intelligence demonstrated by machines, unlike the natural intelligence displayed by humans and animals, which involves consciousness and emotionality. The term “artificial intelligence” is used to describe machines (or computers) that mimic “cognitive” functions that humans associate with the human mind, such as, for example, “learning” and “problem-solving.”

As used herein, the term “machine learning (ML)” as used hereinbelow is a study of computer algorithms that configured to improve automatically based on a received. ML is a subset of artificial intelligence. Machine learning algorithms build a mathematical model based on sample data, known as “training data,” in order to make predictions or decisions without being explicitly programmed to do so.

The term “Sudden infant death syndrome (SIDS)” as used hereinbelow is also known as cot death or crib death. SIDS is the sudden unexplained death of a child of less than one year of age. SIDS may occur during the deep sleep and REM stages of an infant.

The term “Sudden and Unexpected Infant Deaths (SUIDs) may include 80% of Sudden Infant Death Syndrome (SIDS), and the other 20% cases are often caused by infections, genetic disorders, and heart problems.

As used herein with embodiments of the discloser, the sleep time of an infant may be divided into at least two broad types: non-Rapid Eye Movement (non-REM or NREM) sleep and Rapid Eye Movement (REM) sleep. Non-REM sleep may occur after a transitional period that also called slow-wave sleep and/or deep sleep. During this phase, the body temperature and heart rate of the infant fall, and the brain may use less energy.

The NREM may be divided into three stages: N1, N2, and N3. Stage N3 may also be called deep sleep or slow-wave sleep. The sleep order may be N1 to N2 to N3 back to N2 and REM.

REM sleep is also known as paradoxical sleep, represents a smaller portion of total sleep time. REM sleep is the main occasion for dreams and is associated with desynchronized and fast brain waves, eye movements, loss of muscle tone, and homeostasis suspension.

The REM sleep occurs as a person returns to stage 2 or 1 from a deep sleep. At the deep sleep level, a low blood amount of oxygen may occur. The low blood amount of oxygen may create a critical situation for SIDS, which may lead to brain damage.

During REM sleep, brain activity picks up, nearing levels seen when the infant awake. At the same time, the body experiences atonia, which is a temporary paralysis of the muscles, with two exceptions: the eyes and the muscles that control breathing. Even though the eyes are closed, they can be seen moving quickly.

As described in Table 1 below, Non-REM sleep consists of several different sleep stages, e.g., N1 , N2, and N3, each characterized by a specific pattern of brain activity reflecting sleep depth. N1, N2, N3, and REM may occur across a cycle of 50 minutes for infants and toddlers and 90-110 minutes for older children and adults, repeating multiple times throughout the night.

For example, the N1 sleep stage may be referred to as light sleep, marking the transition from wakefulness to sleep. The electroencephalogram (EEG), e.g., a graphic record of the electrical activity of the brain, of N2 sleep stage mainly occurs across a frequency range of 4-7 Hz in the theta waveband, and is accompanied by a similar decrease in muscle tone as in N1, as well as reduced heart rate, lowered core body temperature, and no eye movements.

A K-complex is a high amplitude negative wave, followed by a high voltage positive slow-wave, first occurring after six months of age. Sleep spindles are spurts of high amplitude, high-frequency waves, first occurring after four weeks of age. Sleep spindles may be caused by interactions between neurons in the brain, specifically around the areas of the thalamus and cortex. They operate at varying frequencies between 9-16 Hz. However, sleep spindles mainly occur at 12-14 Hz.

For example, the N3 sleep stage is considered to be the deepest stage of sleep. The EEG of N3 consists of high amplitude, low-frequency waves of brain activity, mainly in the delta waveband (0-4 Hz). Due to these large, slow waves, N3 may be referred to as Slow Wave Sleep (SWS). Whilst the EEG of N3 is characteristically different from that of N2, similarities occur in the occasional presence of sleep spindles and decreased heart rate and core body temperature. Contrasting to earlier stages of sleep, N3 may have a high arousal threshold, meaning that it is difficult to wake someone from this stage of sleep. The amount of time spent in N3 is greatest in early childhood, reducing in length as we grow older.

The characteristic of sleep stages is show with Table 1.

TABLE 1
Characteristics of Sleep Stage EEG

| | EEG | EEG | | |
|---|---|---|---|---|
| | Frequency | Ampli- | Wave- | |
| Stage | Rate (Hz) | tude | band | Associated Characteristics |
| N1 | 6-8 Mixed | Low | Theta | Low arousal threshold |
| | frequency | | | Slow, rolling eye movements |
| | | | | A decrease in muscle tone |
| | | | | Thoughts lost logical |
| | | | | coherence |
| N2 | 4-7 Sleep | Medium | Theta | Sleep spindles |
| | spindles: | | | K-complexes |
| | 9-16 | | | A decrease in muscle tone |
| | K-complexes: | | | Reduced heart rate |
| | 0.5-2 | | | Lowered body temperature |
| | Low frequency | | | |
| N3 | 1-4 Low | | Delta | Occasional sleep spindles |
| | frequency | | | High arousal threshold |
| | | | | Decreased heart rate |
| | | | | Lowered body temperature |
| REM | Above 8 Hz | Low | Alpha/ | Rapid eye movements |
| | Mixed | | Beta | Muscle atonia |
| | frequency | | | |

In some demonstrative embodiments, the sensors that may measure vital signs of the infant may be operated in a reflective mode, a transmissive mode, and/or any other desired mode.

For example, the reflective mode may be used in cases where one or more signals from different deeps of the tissue may require that a light source and a sensor are placed in the same plan. The light may penetrate the tissue, and a portion of the light may be reflected back to the sensor.

For example, a transmissive mode may be used for Heart Rate Monitor (HRM) and Oxygen Saturation (SpO2). A photoplethysmogram (PPG) signal may be used in this mode because it is less sensitive to the risk of saturation, and the PPG signal-to-noise ratio (SNR) may be higher.

In this demonstrative embodiment, the light source may be located opposite to the sensor, and the light may travel all along with the tissue and is captured in the other face by the sensor.

In some demonstrative embodiments, medical SpO2 finger clips may use the transmissive mode, while most of the wearables devices, such as, for example, smartwatches, wristbands, earbuds and etc. may use the reflective mode to monitor the sleep stages by using a combination of the infant movement and heart-rate patterns.

In some demonstrative embodiments, an algorithm may be used to predict the infant sleep stages. For example, the algorithm may compare the sleep pattern of the infant to a monitored sleep pattern of the infant to define the sleep stage of the infant. For example, the algorithm may detect that the infant is asleep based on the moving pattern of the infant, a heart rate, and a historical sleep pattern of the infant.

Furthermore, the length of time of the movements of the infant may indicate certain sleep behavior, which confirms that sleeping.

For example, while the infant is sleeping, the algorithm may track beat-to-beat changes in the heart rate of the infant. The algorithm may also be known as a heart rate variability (HRV) algorithm.

In some demonstrative embodiments, the HRV algorithm may monitor vital signs such as, for example, PPG, ECG, GSR signals, SpO2, Body Temp, Sleep monitoring, respiration rate, and a motion of the infant.

For example, the HRV may fluctuate when the infant sleep transition between light sleep to deep sleep and REM sleep stages. The infant's movement and heart rate patterns may be used to estimate his sleep cycles from the previous night to the current night.

In some demonstrative embodiments, the HRV algorithm may be based on Inter-Beat Intervals (IBI)s derived from ECG data.

The advantage of HRV-based, methods is the potential to apply the algorithms on IBI-measurements obtained by non-obtrusive alternatives for ECG.

In some demonstrative embodiments, a wearable device may be worn, for example, on a wrist and/or arm and/or leg of the infant and may include the IBI-based algorithm.

For example, the wearable device may include the PPG sensor to assess the performance of the algorithm on raw PPG data and investigate the effect of direct application of a machine learning approach on a different type of raw data without re-training.

In some demonstrative embodiments, to compute the HRV features, individual heartbeats were detected from the raw PPG signal using a template-based beat segmentation algorithm. The time difference between each pair of heartbeats was calculated, and implausible IBIs with a duration lower than 0.3 sec or higher than 1.5 sec was excluded. Gross body movements were quantified as activity counts for each 30 sec of the recording based on the three-axial accelerometer signal.

In some demonstrative embodiments, an algorithm based on knowledge of human signal analysis to measure vital signs and human body condition on several different physiological noninvasive signals may be used.

For example, the algorithm may be individually tailored to infants, define the risk level, and initiate immediate life rescue actions.

In some demonstrative embodiments, the algorithm may prevent the infant from entering the deep sleep stage in extreme situations by gentle vibrations and adjusted frequency (adjusted to the infant age) which will stimulate the infant brain in extreme situations. For example, the frequency may be in ranges of 20 Hz to 20 KHz. The frequencies may be set based on the age of the infant.

In some demonstrative embodiments, the algorithm may use a predictive AI to analyze data, screen false indications, and/or forecast individual danger levels. It should be understood that the architecture of HRV/PPG/ECG/GSR signals, SpO2, body temperature, sleep monitoring, respiration rate, and motion may be measured from at least one wrist and a leg if desired.

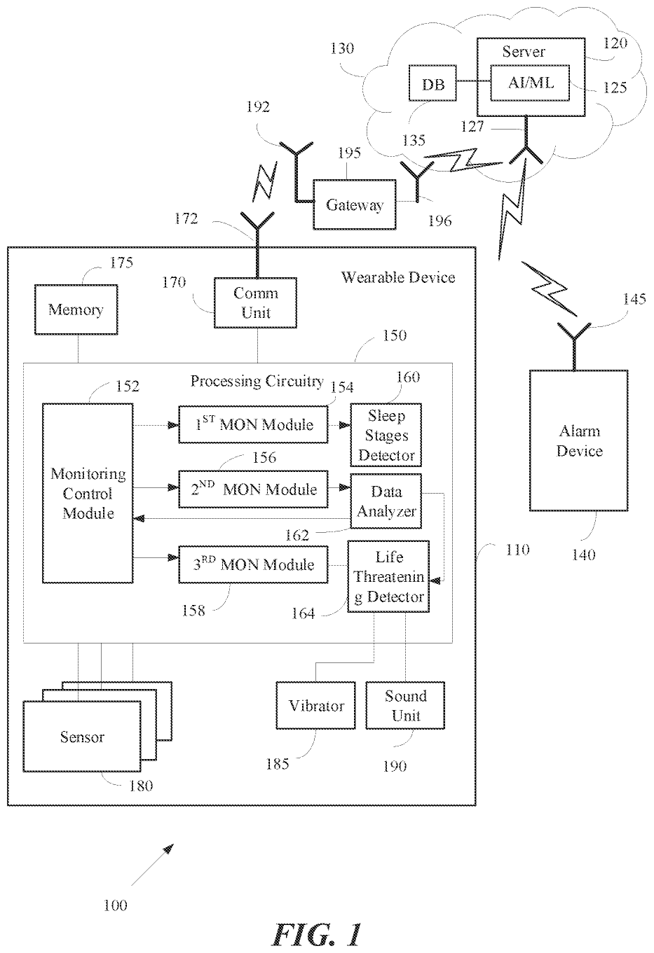

Reference is made first to FIG. 1, which is an illustration of a block diagram of a system 100 to monitor a sleep of an infant, according to some demonstrative embodiments.

In some demonstrative embodiments, system 100 may include a wearable device 110 for monitoring a sleeping behavior of an infant, a server 120 located in a cloud 130, and a database (DB) 135.

In some demonstrative embodiments, server 120 may include a processing circuitry 125, which may be operably coupled to a database 135. For example, database 135 may include a historical database that may store historical measurement and parameters, medical information, historical sleep patterns, and the like, and a current database that may include measurement and parameters of a specific infant. It should be understood that in other embodiments, server 120 may include database 135.

For example, database 135 may include medical theory references and data related to the infant medical record. This data may include past diagnoses, diseases, genetic diseases, personal historical data, age of the parents, mother BMI, high-risk pregnancy, type of birth, mother type of blood, race, and ethnicity, baby birth age in weeks, type of blood, birth weight, Male/Female, sleeping patterns, etc.

In some demonstrative embodiments, processing circuity 125 may use ML to study the infant sleep pattern by comparing vital signs data of the infant to the data stored in database 135, analyze the data and predict the risk profile of the infant to SIDS based on the learning.

For example, the machine learning algorithm may be configured to weight data received from the two or more sensors to generate weighted data; compare a historical medical measurement data of a plurality of infants to the weighted data received from the one or more sensor, and set a risk level of the infant based on the comparison.

Furthermore, for example, the machine learning algorithm may be configured to identify a sleep stage based on the two or more vital signs and predict an entering to a next sleep stage base on the two or more vital signs, wherein the two or more vital signs comprise a heart rate pattern and historical sleep pattern of the infant.

For example, the machine learning algorithm may be configured to predict a life-threatening state of the infant based on historical databased records, one or more vital signs of the infant.

For example, the machine learning algorithm may be configured to analyze data received from the one or more vital signs and from a historical database; and define the risk threshold value based on the learning.

In some demonstrative embodiments, server 120 may include a wireless communication unit (not shown) operably coupled to one or more antennas 127. For example, the communication unit may include a cellular radio, a WiFi radio, and/or a Bluetooth radio and the like.

In some demonstrative embodiments, the one or more antennas 127 may include a dipole antenna, a Yagi antenna, an internal antenna, a monopole antenna, a whip antenna, an antenna array, a phased array antenna, and the like.

In some demonstrative embodiments, system 100 may include a gateway 195 operably coupled to antennas 192 and 196

In some demonstrative embodiments, the one or more antennas 192 an 196 may include a dipole antenna, a Yagi antenna, an internal antenna, a monopole antenna, a whip antenna, an antenna array, a phased array antenna, and the like.

In some demonstrative embodiments, gateway 195 may be configured to wirelessly connect the wearable device 110, e.g., through antennas 192 and 196, to server 120. For example, gateway 195 may use a Bluetooth Low Energy (BLE) to communicate with wearable device 110 and with wireless local area network (WLAN) and or cellular network to communicate with server 120

In some demonstrative embodiments, wearable device 110 may include a communication unit 170. For example, communication unit 170 may include a WiFi radio, a Bluetooth (BT) radio, a cellular radio, a Bluetooth Low Energy (BLE) radio, a universal series bus (USB) interface, a network interface, and the like.

In some demonstrative embodiments, communication unit 170 may be operably connected to antenna 172. For example, antenna 172 may include a dipole antenna, a Yagi antenna, an internal antenna, a monopole antenna, a whip antenna, an antenna array, a phased array antenna, and the like.

In some demonstrative embodiments, wearable device 110 may include a memory 175. For example, memory 175 may include software instructions configured to monitor and predict a sleep stage of an infant and the risk of being in SIDS, an operation system, temporary storage of the sensor reading, and the like.

In some demonstrative embodiments, memory 175 may include Volatile memory, such as, for example, Random-access memory (RAM), Dynamic random-access memory (DRAM), Synchronous dynamic random-access memory (SDRAM), Double data rate (DDR) SDRAM, Static random-access memory SRAM and/or Non-volatile memory (NVM), such as, for example, Read-only memory (ROM), Programmable ROM (PROM), Erasable programmable read-only memory (EPROM), Electrically erasable programmable read-only memory (EEPROM), Flash memory and the like.

In some demonstrative embodiments, wearable device 110 may include processing circuitry 150, a communication unit 170, a memory 175, a plurality of sensors 180.

In some demonstrative embodiments, wearable device 110 may include a sleeve and/or bracelet that may be wear on the wrist and/or a leg of the infant.

In some demonstrative embodiments, the processing circuitry may include, for example, a monitoring control module 152, a first monitoring module 154, a second monitoring module 156, a third monitoring module 158, a data analyzer module 160, sleep stages detector module 162, and a life-threatening detector 164.

In some other demonstrative embodiments, a first monitoring module 154, a second monitoring module 156, a third monitoring module 158 may be embedded in a one monitoring module which may be controlled by a monitoring control module 152.

In some other demonstrative embodiments, first monitoring module 154, the second monitoring module 156, third monitoring module 158 may be the same monitoring module.

In some demonstrative embodiments, the processing circuitry 150 may be configured to determine an infant risk profile for sudden infant death syndrome (SIDS) based on an indication generated by a predictive AI algorithm. For example, the predictive AI algorithm may be performed by processing circuitry 125 of server 120. The processing circuitry 125 may use an ML process that may compare the data stored, e.g., historical data, at the database 135 to an infant monitored life signs data, sleep pattern, heart rate, and the like.

In some demonstrative embodiments, the processing circuitry 15 may be configured to perform in parallel a first periodically monitoring performed, for example, by the first monitoring module 154, having a first monitoring schedule of one or more infant vital signs.

In some demonstrative embodiments, the processing circuitry 150 may be configured to set a risk threshold value by monitoring one or more sensors 180 that are operably coupled to processing circuitry 150 and to determine a deep sleep stage or REM. For example, the detection of deep sleep and/or REM may be done by sleep stages detector 160, based on at least two vital signs.

In some demonstrative embodiments, the processing circuitry 150 may be configured to perform a second periodically monitoring, for example, by second monitoring module 156, having a second monitoring schedule to define an infant risk threshold based on a data analyzer module 162.

In some demonstrative embodiments, at the deep sleep stage, the processing circuitry 150 may be configured to change the second monitoring schedule and update the infant risk threshold value base on the infant risk profile and the one or more vital signs.

In some demonstrative embodiments, when the infant risk threshold value is crossed, the processing circuitry 150 may be configured to perform third monitoring, performed, for example, by third monitoring module 158, at a third monitoring schedule. For example, the third monitoring module 158 may be configured to predict a life-threatening state performed, for example, by a life-threatening detector 164 and processing circuitry 150.

In some demonstrative embodiments, at the life-threatening state, the processing circuitry 150 may be configured to generate one or more signals to prevent the infant from being in the life-threatening state.

In some demonstrative embodiments, the processing circuitry 150 may include a one-core processor, a multi-core processor, one or more processors, a digital signal processor (DSP), and/or any other processing circuitry.

In some demonstrative embodiments, the second monitoring rate is faster than the first monitoring rate, and the third monitoring rate is faster than the second monitoring rate.

In some demonstrative embodiments, the first monitoring rate may be se by the monitoring control module 152 based on an awakened state of the infant.

In some demonstrative embodiments, the two or more vital signs may include at least an infant movement and a heart rate pattern.

In some demonstrative embodiments, processing circuitry 150 may be configured to estimate a sleep stage, e.g., Non-REM, N1, N2, N3, deep sleep, REM, of the infant based on a combination of the movement of the infant and the heart rate pattern of the infant, although it should be understood that this is an example only and other combination of vital signs and predictive AI algorithm may be used to predict the sleep stage.

In some demonstrative embodiments, processing circuitry 150 may be configured to determine the infant risk profile by weighing data received from the two or more sensors 180 based on a machine learning algorithm as described above.

In some demonstrative embodiments, processing circuitry 150 may be configured to compare historical medical measurement data, e.g., received from database 135, of a plurality of infants to the weighted data received from the one or more sensors 180 and to set a risk level of the infant based on the comparison.

In some demonstrative embodiments, processing circuitry 150 may be configured to predict an entering to a next sleep stage base on the two or more vital signs, for example, a combination of the movement of the infant and the heart rate pattern of the infant.

In some demonstrative embodiments, processing circuitry 150 may be configured to analyze data received from the one or more vital signs and from a historical database and to define the risk threshold value based on the machine learning algorithm.

In some demonstrative embodiments, when the infant is in a life-threatening state, vibrator 185 may be configured to vibrate the infant in a frequency range of, for example, 20 Hz to 20 KHz.

In some demonstrative embodiments, when the infant is in a life-threatening state, the sound unit 190 may be configured to transmit a wave, which may be configured to cause brain stimulation to the infant. For example, the frequency of the wave may be determined according to the infant profile.

In some demonstrative embodiment, the wave may include an electromagnetic wave, a sound wave, a low-frequency wave, a tone, or the like.

For example, when the wave includes a tone, the frequency of the tone may vary, for example, at the range of 20 Hz to 20 KHz.

In some demonstrative embodiments, wearable device 110 may include an alarm device 140. The alarm device 140 may be configured to play an alarm signal when the infant is in a life-threatening state.

In some demonstrative embodiments, the communication unit 170 may transmit to the alarm device 140 a notification signal that causes an alarm at the alarm device 140. For example, the alarm device 140 may play an alarm signal and/or send an alert message about the infant being in a life-threatening state.

In some demonstrative embodiments, the alarm device 140 may include a mobile device, a cellular device, a personal assistance device, a home control device, a desktop computer, a laptop computer, a tablet computer, an infant alarm device and etc.

In some demonstrative embodiments, the alarm device 140 may be operably coupled to antenna 145. For example, antenna 145 may include a dipole antenna, a Yagi antenna, an internal antenna, a monopole antenna, a whip antenna, an antenna array, a phased array antenna, and the like.

In some demonstrative embodiments, the second monitoring module 156 may detect that the infant may be turned from laying on his back to his side or to his bally. In this case, the processing circuitry 150 may be configured to send, for example, an alarm message to alarm device 145.

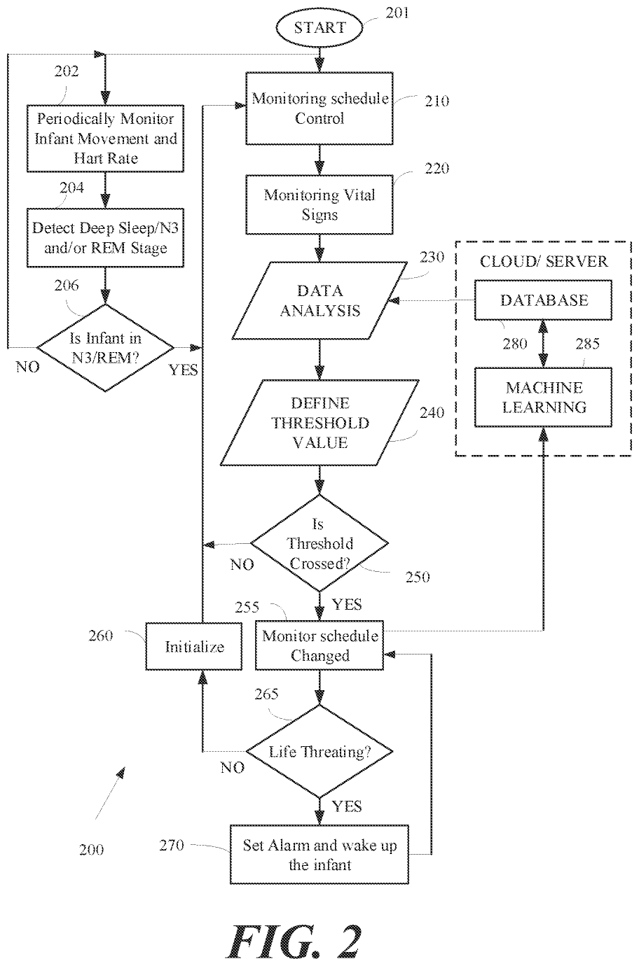

Reference is now made to FIG. 2, which is a schematic illustration of a flow chart of a method 200 to monitor a sleep of an infant, according to some demonstrative embodiments. For example, the method may start (ellipse 201) with two parallel monitoring cycles on the infant sleep stages. The first monitoring may be done by the first monitoring module 154 (FIG. 1), and the second monitoring may be done by the second monitoring module 156 (FIG. 1).

In some demonstrative embodiments, the first monitoring module 154 (FIG. 1) may periodically monitor, e.g., every 30 minutes, at least an infant's movement and a heart rate of the infant (text box 202). Sleep stages detector module 162 (FIG. 1) may detect a deep sleep stage and/or a REM stage (text box 204) and keep monitoring until the infant is in an N3, e.g., deep sleep stage and/or REM stage (diamond 206). When the infant is in the deep sleep stage and/or REM stage (diamond 206), the monitoring control module 152 (FIG. 1) may change the monitoring schedule (text box 210) of the second monitor module (FIG. 1).

In some demonstrative embodiments, the second monitor module (FIG. 1) may monitor the vital signs of the infant (text box 220) at all sleep stages as described in Table 2 and Table 3 below.

In some demonstrative embodiments, the second monitoring schedule of the second monitor module 15 (FIG. 1) may be controlled by the monitoring control module 15 (FIG. 1) (text box 210). For example, the monitoring control module 152 (FIG. 1) may change the monitoring schedule of the second monitor module 156 based on the infant risk, as shown in table 2 and table 3 when monitoring the vital signs (text box 220)

For example, table 2 shows the monitoring schedule of each vital sign of an infant with a low-risk profile. When the infant awake, the monitor may be done every 30 minutes. For example, when the infant sleeps, the monitoring may be done between 1 to 10 minutes. It should be understood that this is an example only, and other monitoring periods may be used with other embodiments.

TABLE 2
Infant at No Risk

| Vital Signs | During Sleep (Min) | Awake (Min) |
|---|---|---|
| Heart Rate | 1 | 30 |
| SP02 | 1 | 30 |
| SKIN TEMP | 10 | 30 |
| Respiration rate | 1 | 30 |
| Motion | 5 | — |
| Sleep Position | 5 | — |
| Microphone | 1 | — |

For example, table 3 shows the monitoring schedule of each vital sign of an infant with a medium to high-risk profile. When the infant awake, the monitoring may be done every 15 minutes, and when the infant sleeps, the monitoring is done every 0.5 to 5 minutes. For example, the setting of the monitoring schedule when the infant is awake may be done by the parent and/or by any other infant supervisor. It should be understood that this is an example only, and other monitoring periods may be used with other embodiments.

TABLE 3
Infant at Medium to High Risk

| Vital Signs | During Sleep (Min) | Awake ((Min) |
|---|---|---|
| Heart Rate | 0.5 | 15 |
| SP02 | 0.5 | 15 |
| SKIN TEMP | 5 | 15 |
| Respiration rate | 0.5 | 15 |
| Motion | 5 | 15 |
| Sleep Position | 5 | — |
| Microphone | 0.5 | — |

In some demonstrative embodiments, the data of the monitoring of the vital signs may be analyzed (rhombus box 230) by a machine learning algorithm (text box 285) based on historical data at the database (text box 280). For example, the database and the machine learning algorithm may be done in a server, e.g., server 120 (FIG. 1), which may be located in the cloud, e.g., cloud 130 (FIG. 1).

In some demonstrative embodiments, the output of the data analysis (rhombus box 230) may be used to define the threshold value (rhombus box 240) for the infant risk to SIDS (rhombus box 240).

For example, to define the threshold value, the machine learning algorithm may compare medical theory references versus historical medical records. The historical medical records may include Past diagnosis, Diseases, Genetic Diseases, Personal Historical data, Parents Ages. Mother BMI, High-Risk Pregnancy, Type of Birth, Mother type of blood, Race, and ethnicity. Baby Birth age and the like.

In some demonstrative embodiments, when the threshold is not crossed (Dimond 250), the vital signs monitoring schedule of the infant may be initialized to a predetermined monitoring schedule (text box 260).

In some demonstrative embodiments, when the threshold is crossed (Dimond 250), the schedule of the monitoring of the infant's vital signs may be (text box 255), for example, based on Table 3.

In some demonstrative embodiments, the second monitoring block, e.g., second monitoring module 156 (FIG. 1), may verify the data analysis result and may identify if the infant is in a life-threatening situation (Dimond 265).

In some demonstrative embodiments, when the second monitoring block, e.g., the second monitoring module 156 (FIG. 1), may identify that the infant is in a life-threatening situation (Dimond 265), then an alarm may set, play sound and vibrate the infant in order to wake up the infante (text box 270).

For example, the frequency of the sound may be within the range of 20 Hz to 20 KHz. The frequency may be adjusted based on the infant's age to cause brain stimulation and awake the infant.

In some demonstrative embodiments, the alarm may be sent to a parent cellular device, an emergency center, and the like.

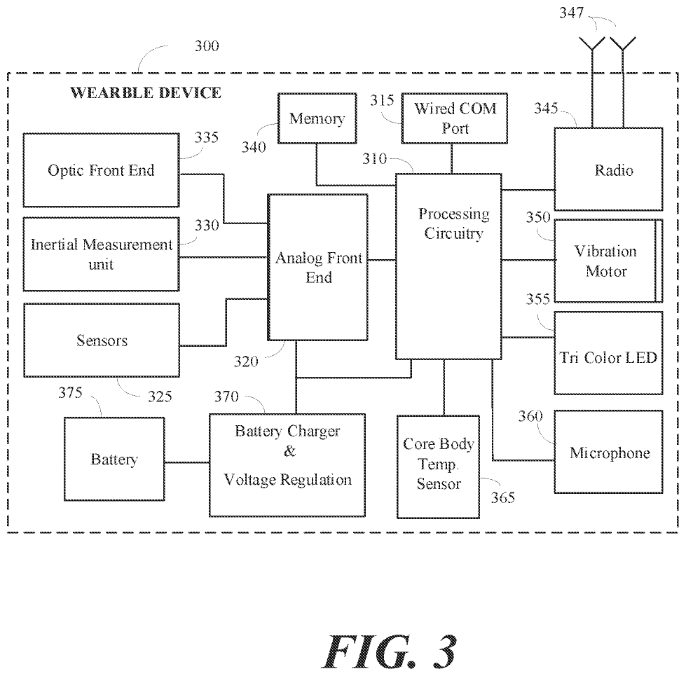

Reference is now made to FIG. 3, which is an illustration of a block diagram of a wearable device 300 configured to monitor a sleep of an infant, according to some demonstrative embodiments.

In some demonstrative embodiments, wearable device 300 may include a processing circuitry 310 configured to operate an AI algorithm, e.g., as described above, to predict a life-threatening situation of an infant as described above with the method of FIG. 2.

In some demonstrative embodiments, processing circuitry 310 may include a one-core processor, a multi-core processor, one or more processors, a digital signal processor (DSP), a microcontroller, and/or any other processing circuitry.

In some demonstrative embodiments, wearable device 300 may include a wired communication port 315 for debagging and diagnostic of the wearable device 300.

For example, wired communication port 315 may include a USB port, a Universal Synchronous/Asynchronous Receiver/Transmitter (USART) port, Serial Wire Debug (SWD), and the like

In some demonstrative embodiments, wearable device 300 may include an analog front end 320. For example, the analog front end 320 may be configured to interface analog signals that may be received from one or more sensors 325, an inertial measurement unit 330, and/or optical front end 335 to processing circuity 310 digital inputs.

In some demonstrative embodiments, the inertial measurement unit 330 may be configured to sense the movement of the infant and to send inertial measurement unit 330 data to the processing circuitry 310. The processing circuitry 310 may determent a lying position of the infant based on the inertial measurement unit 330 data. If the infant is lying on its back, the processing circuitry 310 may generate an alert message. For example, the alert message may be sent to alarm device 140 (FIG. 1).

For example, the inertial measurement unit 330 may include a 6 Axis sensor, an accelerometer, a gyroscope, and/or the like.

In some demonstrative embodiments, optical front end 335 may be configured to receive optic signals from one or more optical sensors and convert the optic signals into analog signals. For example, the one or more optical sensors may include a photodiode sensor.

In some demonstrative embodiments, wearable device 300 may include a memory 340. For example, memory 340 may include software instructions configured to monitor and predict a sleep stage of the infant, and the risk of being in SIDS, an operation system, temporary storage of the sensor reading, and the like.

In some demonstrative embodiments, memory 340 may include Volatile memory, such as, for example, Random-access memory (RAM), Dynamic random-access memory (DRAM), Synchronous dynamic random-access memory (SDRAM), Double data rate (DDR) SDRAM, SRAM (Static random-access memory), and/or Non-volatile memory (NVM), such as, for example, Read-only memory (ROM), Programmable ROM (PROM), Erasable programmable read-only memory (EPROM), Electrically erasable programmable read-only memory, (EEPROM), embedded Multi-Media Controller (eMMC), Flash memory and the like

In some demonstrative embodiments, wearable device 300 may include a radio 345. For example, radio 345 may include a WiFi radio, a Bluetooth (BT) radio, a cellular radio, a Bluetooth Low Energy (BLE) radio, and the like.

In some demonstrative embodiments, radio 345 may be operably connected to two or more antennas 347. For example, the two or more antennas 347 may include a dipole antenna, a Yagi antenna, an internal antenna, a monopole antenna, a whip antenna, an antenna array, a phased array antenna, and the like.

In some demonstrative embodiments, wearable device 300 may include a vibration motor 350. For example, the vibration motor 350 may be configured to provide vibration to the infant when the infant is in a life-threatening situation.

In some demonstrative embodiments, wearable device 300 may include a tri-color Light Emitting Diode (LED) 355. For example, the tri-color LED 355 may be used as light source for the optical sensors and/or include a photodiode sensor, if desired.

In some demonstrative embodiments, wearable device 300 may include a microphone 360. For example, microphone 360 may be configured to record infant sounds, e.g., breathing sound, crying sound, coughing sounds, and any other sound of the infant.

In some demonstrative embodiments, wearable device 300 may include a temperature sensor 365. For example, the temperature sensor 365 may be configured to measure the body temperature of the infant.

In some demonstrative embodiments, wearable device 300 may include a battery charger 370. For example, the battery charger 370 may include a voltage regulator, which may be configured to regulate the charging voltage of a battery 375 to the required charging voltage of the battery 375.

In some demonstrative embodiments, battery 375 may include a rechargeable battery, such as, for example, a nickel-cadmium battery, a nickel-metal hydride battery, a Lithium-ion (Li-Io) battery, a lithium-ion polymer (LiPo) battery, and the like.

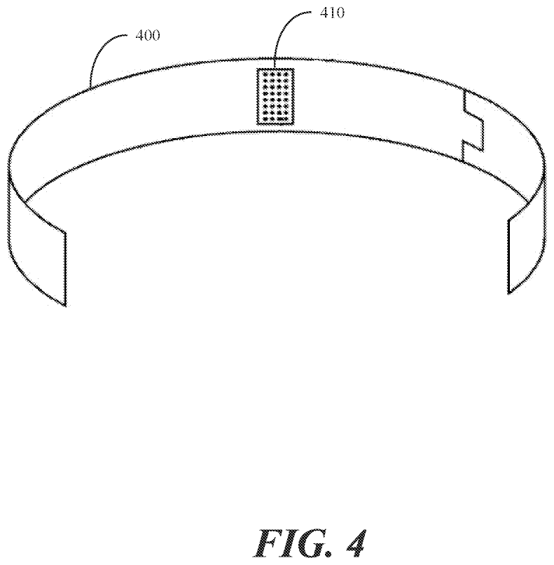

Reference is now made to FIG. 4, which is an illustration of a bracelet 400, which configured to monitor vital signs of an infant, according to some demonstrative embodiments.

In some demonstrative embodiments, the wearable device of FIG. 1 and/or FIG. 3 may include the bracelet 400. However, in other demonstrative embodiments, the wearable device of FIG. 1 and/or FIG. 3 may be included in a diaper, a sleeve, a shirt, pants, or the like.

In some demonstrative embodiments, bracelet 400 may include a wearable device circuitry 410, which has been detailed described in FIG. 1 and/or FIG. 3 above.

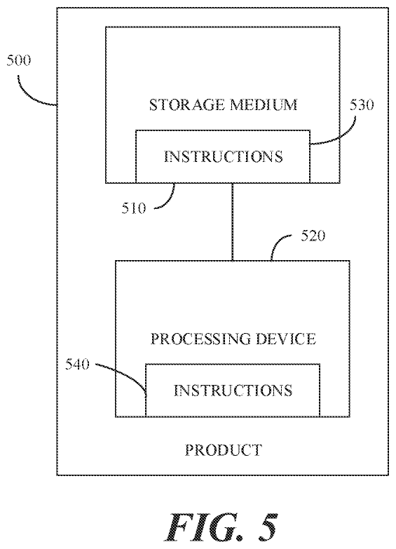

Reference is now made to FIG. 5, which is a schematic illustration of a product of manufacture 500, according to some demonstrative embodiments. Product 500 may include one or more tangible computer-readable non-transitory storage media 510, which may include computer-executable instructions 530, implemented by processing device 520, operable to, when executed by at least one computer processor, enable at least one processing circuitry 150 (FIG. 1) to implement one or more program instructions for monitoring infant sleep stages detect a life-threatening situation and sent alarm and/or awake the infant and/or to perform, trigger and/or implement one or more operations, communications and/or functionalities as described above with reference to FIGS. 1-3. The phrase “non-transitory machine-readable medium” is directed to include all computer-readable media, with the sole exception being a transitory propagating signal.

In some demonstrative embodiments, product 500 and/or machine-readable storage medium 510 may include one or more types of computer-readable storage media capable of storing data, including volatile memory, non-volatile memory, removable or non-removable memory, erasable or non-erasable memory, writeable or re-writeable memory, and the like. For example, machine-readable storage medium 510 may include any type of memory, such as, for example, RAM, DRAM, ROM, programmable ROM (PROM), erasable programmable ROM (EPROM), electrically erasable programmable ROM (EEPROM), Flash memory, a hard disk drive (HDD), a solid-state disk drive (SDD), fusen drive, and the like. The computer-readable storage media may include any suitable media involved with downloading or transferring a computer program from a remote computer to a requesting computer carried by data signals embodied in a carrier wave or other propagation medium through a communication link, e.g., a modem, radio, or network connection.

In some demonstrative embodiments, processing device 520 may include logic. The logic may include instructions, data, and/or code, which, if executed by a machine, may cause the machine to perform a method, process, and/or operations as described herein. The machine may include, for example, any suitable processing platform, computing platform, computing device, processing device, processing circuitry, a computing system, processing system, computer, processor, or the like, and may be implemented using any suitable combination of hardware, software, firmware, and the like.

In some demonstrative embodiments, processing device 520 may include or may be implemented as software, firmware, a software module, an application, a program, a subroutine, instructions, an instruction set, computing code, words, values, symbols, and the like. Instructions 540 may include any suitable types of code, such as source code, compiled code, interpreted code, executable code, static code, dynamic code, and the like. Instructions may be implemented according to a predefined computer language, manner or syntax, for instructing a processor to perform a specific function. The instructions may be implemented using any suitable high-level, low-level, object-oriented, visual, compiled, and/or interpreted programming languages, such as C, C++, C#, Java, Python, BASIC, Mat lab, assembly language, machine code, and the like.

It is to be understood that the system and/or the method for monitoring a sleeping pattern of an infant is described hereinabove by way of example only. Other embodiments may be implemented base on the detailed description and the claims that followed.

It is to be understood that like numerals in the drawings represent like elements through the several figures and that not all components and/or steps described and illustrated with reference to the figures are required for all embodiments or arrangements.

It should also be understood that the embodiments, implementations, and/or arrangements of the systems and methods disclosed herein can be incorporated as a software algorithm, application, program, module, or code residing in hardware, firmware, and/or on a computer useable medium (including software modules and browser plug-ins) that can be executed in a processor of a computer system or a computing device to configure the processor and/or other elements to perform the functions and/or operations described herein.

It should be appreciated that according to at least one embodiment, one or more computer programs, modules, and/or applications that, when executed, perform methods of the present invention need not reside on a single computer or processor but can be distributed in a modular fashion amongst a number of different computers or processors to implement various aspects of the systems and methods disclosed herein.

Thus, illustrative embodiments and arrangements of the present systems and methods provide a computer-implemented method, computer system, and computer program product for processing code(s). The flowchart and block diagrams in the figures illustrate the architecture, functionality, and operation of possible implementations of systems, methods, and computer program products according to various embodiments and arrangements. In this regard, each block in the flowchart or block diagrams can represent a module, segment, or portion of code, which comprises one or more executable instructions for implementing the specified logical function(s).

It should also be noted that, in some alternative implementations, the functions noted in the block can occur out of order noted in the figures. For example, two blocks shown in succession may be executed substantially concurrently, or the blocks can sometimes be executed in the reverse order, depending upon the functionality involved. it will also be noted that each block of the block diagrams and/or flowchart illustration, and combinations of blocks in the block diagrams and/or flowchart illustration, can be implemented by particular purpose hardware-based systems that perform the specified functions or acts or combinations of specialized purpose hardware and computer instructions.

The terminology used herein is to describe particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein are for the purpose of description and should not be regarded as limiting. The use of “including,” “comprising,” or “having,” “containing,” “involving,” and variations thereof herein is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described and without departing from the true spirit and scope of the present invention, which is set forth in the following claims.

What is claimed is:

1. A system for monitoring a sleeping pattern of an infant, comprising a wearable device comprises processing circuitry, wherein the processing circuitry is configured to:
determine an infant risk profile for sudden infant death syndrome (SIDS) based on an indication generated by a predictive Artificial Intelligent (AI) algorithm;
perform in parallel a first periodically monitoring having a first monitoring schedule of one or more infant vital signs to set a risk threshold value, by monitoring one or more sensors that are operably coupled to the wearable device and a second periodically monitoring having a second monitoring schedule to determine a deep sleep stage based on at least two vital signs;
at the deep stage and Rapid Eye Movement (REM) sleep stage, adjust the first monitoring schedule and update the risk threshold value base on the infant risk profile and the one or more vital signs;
when the risk threshold value crossed, perform a third monitoring at a third monitoring schedule to predict a life-threatening state; and
at the life-threatening state, generate one or more signals to prevent the infant from being at the life-threatening state.

2. The system of claim 1, wherein the processing circuitry is configured to determine the infant risk profile based on a machine learning algorithm, wherein the machine learning algorithm is configured to:
weight data received from the two or more sensors to generate weighted data;
compare a historical medical measurement data of a plurality of infants to the weighted data received from the one or more sensor; and
set a risk level of the infant based on the comparison.

3. The system of claim 2, wherein the machine learning algorithm is configured to:
identify a sleep stage based on the two or more vital signs; and
predict an entering to a next sleep stage base on the two or more vital signs, wherein the two or more vital signs comprise a heart rate pattern and historical sleep pattern of the infant.

4. The system of claim 2, wherein the machine learning algorithm is configured to:
predict a life-threatening state of the infant based on historical databased records, one or more vital signs of the infant.

5. The system of claim 2, wherein the machine learning algorithm is configured to:
analyze data received from the one or more vital signs and from a historical database; and
define the risk threshold value based on the learning.

6. The system of claim 1 comprising a server, wherein the server is configured to perform the predictive AI algorithm according to the machine learning algorithm.

7. The system of claim 1, wherein the first monitoring schedule is set based on an awareness state of the infant.

8. The system of claim 1, wherein the two or more vital signs comprise at least an infant movement and a heart rate pattern, and the processing circuitry is configured to:
identify a sleep stage of the infant based on a combination of the movement of the infant and the heart rate pattern of the infant.

9. The system of claim 1, wherein the processing circuitry is configured to:
determine an infant lying position based on an infant movement indication.

10. The system of claim 1, wherein the one or more signals comprise:
a signal configured to vibrate the infant in a low frequency.

11. The system of claim 1, wherein the one or more signals comprise:
a signal configured to cause brain stimulation to the infant.

12. The system of claim 1, wherein the one or more signals comprise:
a signal configured to transmit a wave, wherein the frequency of the wave is determined according to the infant profile.

13. The system of claim 1, wherein the one or more signals comprise:
a signal configured to cause an alarm at an alarm device.

14. A product comprising one or more tangible computer-readable non-transitory storage media comprising program instructions for, wherein execution of the program instructions by one or more processors comprising:
determining an infant risk profile for sudden infant death syndrome (SIDS) based on an indication generated by a predictive Artificial Intelligent (AI) algorithm;
performing in parallel a first periodically monitoring having a first monitoring schedule of one or more infant vital signs to set a risk threshold value, by monitoring one or more sensors that are operably coupled to the wearable device and a second periodically monitoring having a second monitoring schedule to determine a deep sleep stage based on at least two vital signs;
at the deep stage and Rapid Eye Movement (REM) sleep stage, adjusting the first monitoring schedule and update the risk threshold value base on the infant risk profile and the one or more vital signs;
when the risk threshold value crossed, performing a third monitoring at a third monitoring schedule to predict a life-threatening state; and
at the life-threatening state, generating one or more signals to prevent the infant from being at the life-threatening state.

15. The product of claim 14, wherein execution of the program instructions by one or more processors comprises execution of a machine learning program instructions, wherein execution of the machine learning program instructions by one or more processors at a server comprising:
weighting data received from the two or more sensors to generate weighted data;
comparing a historical medical measurement data of a plurality of infants to the weighted data received from the one or more sensor; and
setting a risk level of the infant based on the comparison.

16. The product of claim 15, wherein execution of the machine learning program instructions by one or more processors comprising:
identifying a sleep stage based on the two or more vital signs; and
predicting an entering to a next sleep stage base on the two or more vital signs, wherein the two or more vital signs comprise a heart rate pattern and historical sleep pattern of the infant.

17. The product of claim 15 wherein execution of the machine learning program instructions by one or more processors comprising:
analyzing data received from the one or more vital signs and from a historical database; and
defining the risk threshold value based on the learning.

18. The product of claim 14, wherein execution of the program instructions by one or more processors comprising:
setting the first monitoring schedule based on an awareness state of the infant.

19. The product of claim 14, wherein the two or more vital signs comprise at least an infant movement and a heart rate pattern, and the execution of the program instructions by one or more processors comprises:
identifying a sleep stage of the infant based on a combination of the movement of the infant and the heart rate pattern of the infant.

20. The product of claim 14, wherein execution of the program instructions by one or more processors comprising:
determining an infant lying position based on an infant movement indication.
